(19) United States
(12) Patent Application Publication
(10) Pub. No.: US 2021/0334431 A1
(43) Pub. Date: Oct. 28, 2021

(54) Method for predicting prestress loss after concrete cracking along strand

(71) Applicant: CHANGSHA UNIVERSITY OF SCIENCE AND TECHNOLOGY, Changsha,Hunan (CN)

(72) Inventors: Lei Wang, Changsha, Hunan (CN); Lizhao Dai, Changsha,Hunan (CN); Jianren Zhang, Changsha,Hunan (CN); Xuhui Zhang, Changsha,Hunan (CN); Yafei Ma, Changsha,Hunan (CN)

(21) Appl. No.: 16/492,949

(22) PCT Filed: Apr. 24, 2019
(86) PCT No.: PCT/CN2019/084103

§ 371 Date: Sep. 11, 2019

(30) Foreign Application Priority Data
Jul. 26, 2018 (CN) 201810830975.5

(51) Int. Cl. G06F 30/20; G01N 33/38; G01N 17/00

(52) U.S. Cl. CPC G06F 30/20; G01N 17/00; G01N 33/383

(57) ABSTRACT

The present invention provides a method for predicting prestress loss after concrete cracking along strand. Corrosion-induced cracking is modeled by the thick-wall cylinder theory, the expansive pressure is evaluated with the residual tensile stress by cracked concrete and confining stress by un-cracked concrete during concrete cracking process. Considering the effect of strand corrosion, the bond strength of corroded strand is evaluated from the contributions of adhesion stress, confinement stress and expansive pressure. A method for prestress loss in corroded pre-tensioned concrete member is proposed with the strain compatibility and force equilibrium equations, incorporating the coupling effects of concrete cracking and bond degradation. The present invention proposes a method for predicting the prestress loss after concrete cracking along strand, which can incorporate the coupling effects of concrete cracking and bond degradation. It has a great significance to evaluate the prestress loss in existing pre-tensioned concrete beams.

### CROSS REFERENCE OF RELATED APPLICATION

This is a U.S. National Stage under 35 U.S.C 371 of the international Application PCT/CN2019/084103, filed Apr. 24, 2019, which claims priority under 35 U.S.C. 119(a-d) to CN 201810830975.5, filed Jul. 26, 2018.

### BACKGROUND OF THE PRESENT INVENTION

### Field of Invention

The present invention relates to a technical field of prestress loss evaluation, and more particularly to a method for predicting prestress loss after concrete cracking along strand.

### Description of Related Arts

Prestressed concrete members have long been considered to have good durability. However, due to design defects, poor construction and adverse environmental impact, the performance degradation of prestressed concrete members has become increasingly common. Corrosion will reduce the cross-sectional area of prestressing strand, cause concrete cracking, degrade bond strength, and lead to effective prestress loss, which is one of the main factors for structural durability degradation. A reasonable assessment of the effective prestress in concrete members is a key factor in ensuring their serviceability and safety operation.

The prestress loss of existing concrete members is related to many factors, such as shrinkage and creep of concrete, stress relaxation and corrosion of prestressing strand. Numerous researches have been carried out on the effects of concrete shrinkage and creep, as well as stress relaxation of prestressed steel strands on long-term prestress loss. Some specifications also propose the evaluation methods for long-term prestress loss, such as AASHTO 2010, CEB-FIP 2010. Compared with studies on long-term prestress losses, studies regarding corrosion-induced prestress loss have received little attention. Some scholars used the strain compatibility method to evaluate the residual prestress of post-tensioned concrete beams. Some scholars also pointed out that the residual prestress of post-tensioned concrete beams can be estimated by the residual cross-sectional area of corroded strand. The conventional research mainly analyzes the influence of the corroded strand cross-sectional area reduction on the prestress loss of post-tensioned concrete members. The strand corrosion-induced prestress loss is a very complicated problem. Except for the cross-section reduction of corroded strand, concrete cracking and bond degradation can also cause prestress loss. Additionally, post-tensioned concrete members use the anchorage systems to transmit prestress, whereas prestress in pre-tensioned concrete structures is built through the bond stress at the strand-concrete interface. Concrete cracking and bond degradation may have a more significant impact on prestress loss in pre-tensioned concrete structures than that in post-tensioned concrete members. The research on prestress loss in corroded pre-tensioned concrete members has not been reported yet. How to reasonably evaluate the prestress loss in corroded pre-tensioned concrete members needs further study.

Therefore, the present invention proposes a method for predicting the prestress loss after concrete cracking along strand. The superiority of this method is that it can incorporate the coupling effects of concrete cracking and bond degradation on the prestress loss in pre-tensioned concrete members.

### SUMMARY OF THE PRESENT INVENTION

An object of the present invention is to provide a method for predicting prestress loss after concrete cracking along strand, which can reasonability evaluate prestress loss in corroded pre-tensioned concrete members.

In order to accomplish the above object, the present invention provides:

a method for predicting prestress loss after concrete cracking along strand, comprising steps of:

(1) predicting corrosion-induced concrete cracking, specifically comprising: determining geometric parameters according to specimens details; simulating corrosion-induced cracking by a thick-wall cylinder theory; predicting expansive pressure with the residual tensile stress by cracked concrete and confining stress by un-cracked concrete during concrete cracking process;

(2) analyzing bond strength degradation of corroded strand, specifically comprising: establishing expressions of the adhesion stress, confinement stress and expansive pressure at strand-concrete interface; considering the influence of strand corrosion on the above factors, and then calculate the bond strength of corroded strand;

(3) evaluating corrosion-induced prestress loss, specifically comprising: discretizing the pre-tensioned concrete member into several segments to analyze the stress variation in corroded strand; considering the effects of concrete cracking and bond degradation, the effective prestress in corroded pre-tensioned concrete member is evaluated with the strain compatibility and force equilibrium equations, and then the corrosion-induced prestress loss is obtained.

Preferably, in the step (1), during the corrosion-induced concrete cracking process, the expansive pressure is calculated as:

before cover cracking, the expansive pressure would be balanced by the residual tensile stress by cracked concrete and the confining stress by un-cracked concrete; the expansive pressure P_{c} at the strand-concrete interface is expressed as an equation (1):

P_{c}R₀=PᵤRᵤ+∫_{R}_{₀}^{R}^{ᵤ}σ_{θ}(r)dr

where R₀ is the radius of un-corroded wire, Pᵤ is the expansive pressure at the interface between cracked and un-cracked regions, Rᵤ is the radius of cracked region, r is the position of cracked region, and σ_{θ}(r) is the hoop stress of cracked concrete;

after cover cracking; the expansive pressure would be resisted by the residual tensile strength by cracked concrete; the expansive pressure P_{c} at the strand-concrete interface is expressed as an equation (2):

P_{c}R₀=∫_{R}_{₀}^{R}^{_{c}}σ_{θ}(r)dr.

Preferably, in the step (2), the bond strength of corroded strand is calculated as:

the bond strength of corroded strand is attributed to adhesion stress, friction stress, and expansive pressure at strand-concrete interface, which is expressed as an equation (3):

τ_{η}=τₐ+τ_{b}+τ_{c}

where τ_{η} is the bond stress of corroded strand, τₐ is the bond stress induced by expansive pressure, τ_{b} is the adhesion stress of corroded strand, and τ_{c} is the confinement stress from the surrounding concrete;

The bond stress induced by expansive pressure is expressed as an equation (4):

τₐ=k_{c}p_{c}

where k_{c} is the friction coefficient between corroded strand and cracked concrete;

The adhesion stress of corroded strand is expressed as an equation (5):

$${τ}_{b} = \frac{{kA}_{r} [ cot δ + tan ( δ + θ ) ]}{π {D}_{{S}_{r}}} {f}_{coh}$$

where k is the number of transverse ribs, Aᵣ is the rib area in the plane at right angles to strand axis, D is the strand diameter, δ is the rib orientation, θ is the friction angle between strand and concrete, sᵣ is the rib spacing, and f_{coh} is the coefficient of adhesion stress;

The confinement stress from the surrounding concrete can be given as an equation (6):

$${τ}_{c} = \frac{{kC}_{r} tan ( δ + θ )}{π} {p}_{x}$$

where Cᵣ is the shape factor constant of the transverse ribs, and pₓ is the maximum pressure at bond failure.

Preferably, in the step (3), the effective prestress in corroded pre-tensioned concrete member is calculated as:

one half of the beam is discretized into several segments from 1 to n to analyze the stress variation in corroded strand, for an arbitrary segment i, the stress of corroded strand f_{p,i} can be written as an equation (7):

f_{p,i}=f_{p,i+1}−Δf_{p,i}

where Δf_{p,i} is the local stress variation in the corroded strand at segment i, 1≤i≤n;

The local stress variation in the corroded strand Δf_{p,i} at segment i is given as an equation (8):

$$Δ {f}_{p , i} = \frac{8 π {R}_{ρ , i}}{{A}_{p , i} ( η )} {τ}_{η} {l}_{i}$$

where lᵢ is the segment length, A_{p,i}(η) is the residual cross-sectional area of corroded strand at segment i, and R_{ρ,i} is the residual radius of corroded wire at segment i;

for corroded pre-tensioned concrete structures, the strand prestress at the beam end is zero, i.e., f_{p,i}=0; the tensile stress f_{p,i} of corroded strand at segment i is expressed as an equation (9):

$${f}_{p , i} = {\sum }_{1}^{i - 1} Δ {f}_{p , i - 1}$$

The tension force T_{p,i} of the corroded strand at segment i is expressed as an equation (10):

T_{p,i}=f_{p,i}A_{p,i}(η)

After corrosion, the strand strain change Δε_{p,i} at segment i is expressed as an equation (11):

$${Δɛ}_{p , i} = \frac{{T}_{pi}}{{E}_{p} {A}_{p}} - \frac{{T}_{p , i}}{{E}_{p} {A}_{p , i} ( η )}$$

where Tₚᵢ is the prestressing force of the un-corroded strand at segment i, and Eₚ is the elastic modulus of strand;

The internal stress of corroded strand gradually increases along the member direction until the effective prestress is reached; when the stress of the corroded strand reaches the effective prestress, the concrete strain change Δε_{c,i} at the strand position should be equal to the strand strain change Δε_{p,i} so that strain compatibility is maintained, which is expressed as an equation (12):

Δε_{c,i}=Δε_{p,i}

when the stress of corroded strand reaches the effective prestress, the forces of prestressing strand, concrete and steel reinforcements should satisfy the equilibrium equation (13):

Cᵢ+F′_{s,i}−T_{p,i}−F_{s,i}=0

where Cᵢ is the total force of concrete at segment i, and F_{s,i} and F′_{s,i} are the forces of steel reinforcements in the tension and compression zones at segment i, respectively;

considering the effects of concrete cracking and bond degradation factors, the effective prestress in corroded pre-tensioned concrete member is calculated with the strain compatibility and force equilibrium equations, and then the corrosion-induced prestress loss can be evaluated.

Beneficial effects of the present invention are that: the present invention provides a method for predicting prestress loss after concrete cracking along strand, corrosion-induced cracking is modeled by the thick-wall cylinder theory, the expansive pressure is evaluated with the residual tensile stress by cracked concrete and confining stress by un-cracked concrete during concrete cracking process; considering the influence of strand corrosion, the bond strength of corroded strand is evaluated from the contributions of adhesion stress, confinement stress and expansive pressure; a method for prestress loss in corroded pre-tensioned concrete member is proposed, incorporating the coupling effects of concrete cracking and bond degradation; The present invention proposes a method for predicting the prestress loss after concrete cracking along strand, the superiority of this method is that it can incorporate the coupling effects of concrete cracking and bond degradation on the prestress loss, and can reasonably evaluate the prestress loss after concrete cracking; the calculation result is reliable, which can be widely used in engineering practice.

In order to more clearly illustrate the features and effects of the present invention, the present invention will be described in detail below with reference to the accompanying drawings and specific embodiments.

### BRIEF DESCRIPTION OF THE DRAWINGS

FIG. 1 is a sketch view of concrete cracking caused by strand corrosion of the present invention;

FIG. 2 illustrates internal stress changes of corroded strand of the present invention;

FIG. 3 illustrates strain distribution in the cross-section of member of the present invention;

FIG. 4 is a flow chart of corrosion-induced prestress loss calculation of the present invention;

FIG. 5 illustrates detailed dimension of test beams of the present invention;

FIG. 6 is a sketch view of the four-point bending load test of the present invention;

FIG. 7 (a) illustrates predicted and tested values of effective prestress for group A of the present invention;

FIG. 7 (b) illustrates predicted and tested values of effective prestress for group B of the present invention.

### DETAILED DESCRIPTION OF THE PREFERRED EMBODIMENT

The present invention provides a method for predicting prestress loss after concrete cracking along strand, comprising steps of:

(1) predicting corrosion-induced concrete cracking, specifically comprising: determining geometric parameters according to specimens details; simulating corrosion-induced cracking by the thick-wall cylinder theory; predicting expansive pressure with the residual tensile stress by cracked concrete and confining stress by un-cracked concrete during concrete cracking process;

(2) analyzing bond strength degradation of corroded strand, specifically comprising: establishing expressions of the adhesion stress, confinement stress and expansive pressure at strand-concrete interface; considering the influence of strand corrosion on the above factors, and then calculate the bond strength of corroded strand;

(3) evaluating corrosion-induced prestress loss, specifically comprising: discretizing the pre-tensioned concrete member into several segments to analyze the stress variation in corroded strand; considering the effects of concrete cracking and bond degradation, the effective prestress in corroded pre-tensioned concrete member is evaluated with the strain compatibility and force equilibrium equations, and then the corrosion-induced prestress loss is obtained.

in the step (1), during the corrosion-induced concrete cracking process, the expansive pressure is calculated as follows.

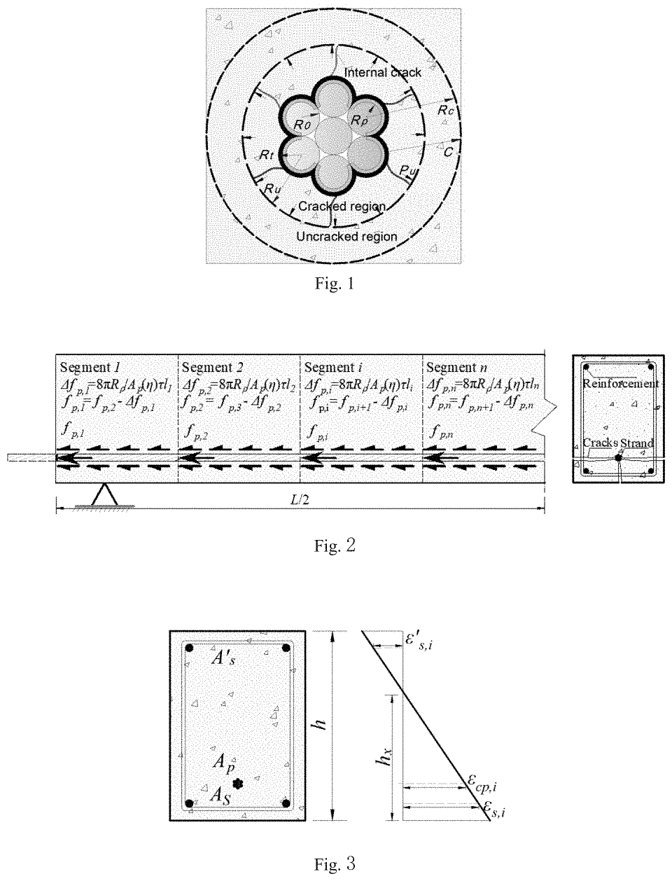

A 7-wire steel strand is taken as the research object. The outer wires would be first corroded when the strand is under environmental erosion, as shown in FIG. 1. Assuming that the corroded region is ⅔ circumference of a single external wire, then the area loss of single external wire is

$$\frac{2}{3} π ( {R}_{0}^{2} - {R}_{ρ}^{2} ) ,$$

and the corrosion loss p of strand can be expressed as an equation (1):

ρ=4π(R₀²−R_{ρ}²)/Aₚ

where R₀ and R_{ρ} are the radiuses of wire before and after corrosion, respectively, and Aₚ is a cross-sectional area of a un-corroded strand.

Corrosion products have larger volume than the consumed iron, and may expand outward. Some corrosion products fill pores and cracks, and others contribute to expansive pressure. By the volume equivalent principle, the volume reduction of corroded strand per unit length can be expressed as an equation (2):

ΔVₜ=ΔV_{w}+ΔVₑ+ΔV_{c}

where ΔVₜ is the total volume of corrosion products per unit length, ΔVₜ=nΔV_{w}, n is the rust expansion ratio, which is set as 3, ΔV_{w} is the volume reduction of corroded wire per unit length,

$$Δ {V}_{w} = \frac{2}{3} π ( {R}_{0}^{2} - {R}_{ρ}^{2} ) ,$$

ΔVₑ is the concrete volume change per unit length,

$$Δ {V}_{e} = \frac{2}{3} π ( {R}_{t}^{2} - {R}_{0}^{2} ) ,$$

Rₜ is the radius of the wire with corrosion products, and ΔV_{c} is the volume of corrosion products per unit length that fill in cracks and pores.

The volume of corrosion products per unit length that in cracks and pore can be expressed as an equation (3):

$$Δ {V}_{c} = \frac{2}{3} π ( {R}_{t} - {R}_{0} ) ( {R}_{u} - {R}_{t} )$$

where Rᵤ is the radius of the cracked concrete.

Combing equations (1-3), the concrete displacement u_{c} induced by expansive pressure can be given as an equation (4):

$${u}_{c} = {R}_{t} = {R}_{0} = \frac{( n - 1 ) {A}_{p} ρ}{4 π ( {R}_{u} + {R}_{0} )}$$

Before cover cracking, the concrete cover consists of two regions: a cracked inner region and an un-cracked outer region, as shown in FIG. 1. For the un-cracked concrete outer region, concrete internal stress can be simulated by an elastic theory. The hoop stress σ_{θ}(t) and radial displacement u(t) of un-cracked concrete can be expressed as equations (5) and (6), respectively:

$${σ}_{θ} ( t ) = \frac{{R}_{u}^{2} {P}_{u}}{( {R}_{c}^{2} - {R}_{u}^{2} )} ( 1 + \frac{{R}_{c}^{2}}{{t}^{2}} )$$

$$u ( t ) = \frac{{R}_{u}^{2} {P}_{u}}{{E}_{c} ( {R}_{c}^{2} - {R}_{u}^{2} )} [ ( 1 + {v}_{c} ) \frac{{R}_{c}^{2}}{t} + ( 1 - {v}_{c} ) t ]$$

where t is the radius of the concrete in the un-cracked region, Rᵤ≤t≤R_{c}, R_{c}=Rₒ+C, C is the thickness of concrete cover, Pᵤ is the expansive pressure at the interface between cracked and un-cracked regions, and E_{c} and v_{c} are the elastic modulus and Poisson's ratio of concrete, respectively.

With the stress distribution equivalence principle, the tensile stress of concrete at the interface between cracked and un-cracked regions should be equal to the tensile strength of concrete, i.e. σ_{θ}(Rᵤ)=fₜ. The expansive pressure Pᵤ at the interface between cracked and un-cracked regions can be expressed as an equation (7):

$${P}_{u} = {f}_{t} \frac{{R}_{c}^{2} - {R}_{u}^{2}}{{R}_{c}^{2} + {R}_{u}^{2}}$$

Combing equations (6-7), the radial displacement u(t) of un-cracked concrete can be obtained. It is assumed that the radial displacement u(r) of cracked concrete still satisfies the linear distribution principle, which can be expressed as an equation (8):

$$u ( r ) = \frac{{f}_{t} {R}_{u}^{2}}{{E}_{c} ( {R}_{c}^{2} + {R}_{u}^{2} )} [ ( 1 + {v}_{c} ) \frac{{R}_{c}^{2}}{r} + ( 1 - {v}_{c} ) r ]$$

where r is the radius of the concrete in the cracked region, R₀≤r≤Rᵤ.

Considering the softening behavior of tensile strength of cracked concrete, the hoop stress can be written as an equation (9):

$${σ}_{θ} ( r ) = \{ \begin{matrix}{E}_{c} {ɛ}_{θ} ( r ) , & {ɛ}_{θ} ( r ) \leq {ɛ}_{ct} \\ {f}_{t} [ 1 - 0.85 \frac{{ɛ}_{θ} ( r ) - {ɛ}_{ct}}{{ɛ}_{1} - {ɛ}_{ct}} ] , & {ɛ}_{ct} < {ɛ}_{θ} ( r ) \leq {ɛ}_{1} \\ 0.15 {f}_{t} [ \frac{{ɛ}_{u} - {ɛ}_{θ} ( r )}{{ɛ}_{u} - {ɛ}_{1}} ] , & {ɛ}_{1} < {ɛ}_{θ} ( r ) \leq {ɛ}_{u}\end{matrix}$$

where σ_{θ}(r) and ε_{θ}(r) are the hoop stress and strain of cracked concrete, respectively, ε_{ct} and ε₁ are the strains corresponding to the concrete tensile strength and 15% of concrete tensile strength, and εᵤ is the ultimate strain of concrete.

Before cover cracking, the expansive pressure P_{c} at strand-concrete interface will be balanced by the residual tensile stress by cracked concrete and the confining stress by un-cracked concrete, which is expressed as an equation (10):

P_{c}R₀=PᵤRᵤ+∫_{R}_{₀}^{R}^{ᵤ}σ_{θ}(r)dr

When the crack extends to the concrete surface, the radius of the cracked zone is equal to the thickness of concrete cover, i.e. Rᵤ=R_{c}. After cover cracking, the expansive pressure would be resisted by the residual tensile strength by cracked concrete, which is expressed as an equation (11):

P_{c}R₀=∫_{R}_{₀}^{R}^{_{c}}σ_{θ}(r)dr

In the step (2), the bond strength of corroded strand is calculated as:

the bond strength of corroded strand is attributed to adhesion stress, friction stress, and expansive pressure at strand-concrete interface, which is expressed as an equation (12):

τ_{η}=τₐ+τ_{b}+τ_{c}

where τ_{η} is the bond stress of corroded strand, τₐ is the bond stress induced by expansive pressure, τ_{b} is the adhesion stress of corroded strand, and τ_{c} is the confinement stress from the surrounding concrete;

The bond stress induced by expansive pressure can be written as an equation (13):

τₐ=k_{c}p_{c}

where k_{c} is the friction coefficient between corroded strand and cracked concrete, k_{c}=0.37−0.26(x−x_{cr}), x is the corroded depth of strand, and x_{cr} is the critical corrosion depth of strand at cover cracking.

The adhesion stress of corroded strand is expressed as an equation (14);

$${τ}_{b} = \frac{k {A}_{r} [ cot δ + tan ( δ + θ ) ]}{π D {s}_{r}} {f}_{c o h}$$

where k is the number of transverse ribs, k=2, Aᵣ is the rib area in the plane at right angles to strand axis, Aᵣ=0.07πD², D is the strand diameter, δ is the rib orientation, δ=45°, θ is the friction angle between strand and concrete, tan(δ+θ)=1.57−0.785x, sᵣ is the rib spacing, sᵣ=0.6D, and f_{coh} is the coefficient of adhesion stress, f_{coh}=2−10(x−x_{cr}),

The confinement stress from the surrounding concrete can be given as an equation (15):

$${τ}_{c} = \frac{k {C}_{r} tan ( δ + θ )}{π} {p}_{x}$$

where Cᵣ is the shape factor constant of the transverse ribs, Cᵣ=0.8, and pₓ is the maximum pressure at bond failure.

In the step (3), the effective prestress in corroded pre-tensioned concrete members is calculated as:

one half of the beam is discretized into several segments from 1 to n to analyze the stress variation in corroded strand, FIG. 2 illustrates internal stress changes of corroded strand, and for an arbitrary segment i, the stress of corroded strand f_{p,i} is expressed as an equation (16):

f_{p,i}=f_{p,i+1}−Δf_{p,i}

where Δf_{p,i} is the local stress variable in corroded strand at segment i, 1≤i≤n.

The local stress variation in the corroded strand Δf_{p,i} at segment i is given as an to equation (17):

$$Δ {f}_{p , i} = \frac{8 π {R}_{ρ , i}}{{A}_{p , i} ( η )} {τ}_{η} {l}_{i}$$

where lᵢ is the segment length, A_{p,i}(η) is the residual cross-sectional area of the corroded strand at segment i, and R_{ρ,i} is the residual radius of corroded wire at segment i.

for corroded pre-tensioned concrete structures, the strand prestress at the beam end is zero, i.e. f_{p,1}=0; the tensile stress f_{p,i} of corroded strand at segment i is expressed as an equation (18):

$${f}_{p , i} = {\sum }_{i}^{i - 1} Δ {f}_{p , i - 1}$$

The tension force T_{p,i} of the corroded strand at segment i is expressed as an equation (19):

T_{p,i}=f_{p,i}A_{p,i}(η)

After corrosion, the strand strain change Δε_{p,i} is expressed as an equation (20):

$$Δ {ɛ}_{p , i} = \frac{{T}_{p i}}{{E}_{p} {A}_{p}} - \frac{{T}_{p , i}}{{E}_{p} {A}_{p , i} ( η )}$$

where Tₚᵢ is the prestressing force of the un-corroded strand at segment i, and Eₚ is the elastic modulus of strand.

The internal stress of corroded strand gradually increases along the member direction until the effective prestress is reached; when the stress of the corroded strand reaches the effective prestress, the concrete strain change Δε_{c,i} at the strand position should be equal to the strand strain change Δε_{p,i} so that strain compatibility is maintained, which is expressed as an equation (21):

Δε_{c,i}=Δε_{p,i}

the concrete strain ε_{cp,i} at the position of corroded strand can be expressed as an equation (22):

$${ɛ}_{c p , i} = \frac{{T}_{p i}}{{E}_{c}} ( \frac{1}{A} + \frac{{e}_{p}^{2}}{I} ) - Δ {ɛ}_{c , i}$$

where eₚ is an eccentricity of strand, A is the cross-sectional area of intact concrete, and I is the inertia moment of the gross section of intact concrete.

The present invention primarily investigates the prestress loss caused by strand corrosion, and no corrosion is considered in steel reinforcements. FIG. 3 illustrates the strain distribution in the beam cross-section. The strains of the ordinary bars in the tension zone and the compression zone, ε_{s,i} and ε′_{s,i}, respectively, are expressed as equations (23) and (24):

$${ɛ}_{s , i} = \frac{{h}_{x} - {a}_{s}}{{h}_{x} - {a}_{p}} {ɛ}_{cp , i}$$

$${ɛ}_{s , i}^{′} = \frac{h - {h}_{x} - {a}_{s}^{′}}{{h}_{x} - {a}_{p}} {ɛ}_{c p , i}$$

where h is the height of beam, hₓ, aₚ and aₛ are the distances from the centers of gravity of beam, strand and tensile reinforcements to the bottom of beam, respectively, and a′ₛ is the distance from the center of gravity of compressive reinforcements to the top of beam.

The stress-strain behavior of steel reinforcements can be described by an elastic-plastic constitutive model, which can be expressed as an equation (25):

$${f}_{s} = \{ \begin{matrix}{E}_{s} {ɛ}_{s} & {ɛ}_{s} \leq {ɛ}_{sy} \\ {f}_{s y} + {E}_{s p} ( {ɛ}_{s} - {ɛ}_{s y} ) & {ɛ}_{s} > {ɛ}_{sy}\end{matrix}$$

where fₛ and f_{sy} are the stress and yield strength of steel reinforcements, respectively, εₛ and ε_{sy} are the strain and yield strain of steel reinforcements, respectively, and Eₛ and Eₛₚ are the elastic and hardening modulus of steel reinforcements, respectively.

The forces of steel reinforcements in the tension and compression zones, F_{s,i} and respectively, at segment i are expressed as equations (26) and (27), respectively:

F_{s,i}=Aₛfₛ(ε_{s,i})

F′_{s,i}=A′ₛfₛ(ε′_{s,i})

where Aₛ and fₛ(ε_{s,i}) are the section area and stress of steel reinforcements in the tension zones, respectively, and A′ₛ and fₛ(ε′_{s,i}) are the section area and stress of steel reinforcements in the compression zones, respectively.

The mechanical behavior of concrete in tension can be modeled by a linear elastic constitutive law. The nonlinear constitutive law of concrete is used to describe the mechanical behavior of concrete in compression. The stress-strain curves of concrete are expressed as an equation (28):

$${f}_{c} = \{ \begin{matrix}{f}_{c}^{′} [ 2 ( \frac{{ɛ}_{c}}{{ɛ}_{0}} ) - {( \frac{{ɛ}_{c}}{{ɛ}_{0}} )}^{2} ] & in compression \\ {E}_{c} {ɛ}_{c} & in tension\end{matrix}$$

where f_{c} and ε_{c} are the stress and strain of concrete, respectively, f′_{c} is the concrete compressive strength, and ε₀ is the concrete strain corresponding to the compressive strength and is set to 0.002.

The total force of concrete C₁ at segment i can be expressed as an equation (29):

Cᵢ=∫_{A}_{_{c}}f_{c}dA_{c}

where A_{c} is the cross-sectional area of damaged concrete.

when the stress of the corroded strand reaches the effective prestress, the forces of prestressing strand, concrete and steel reinforcements should satisfy the equilibrium equation (30):

Cᵢ+F′_{s,i}−T_{p,i}−F_{s,i}=0

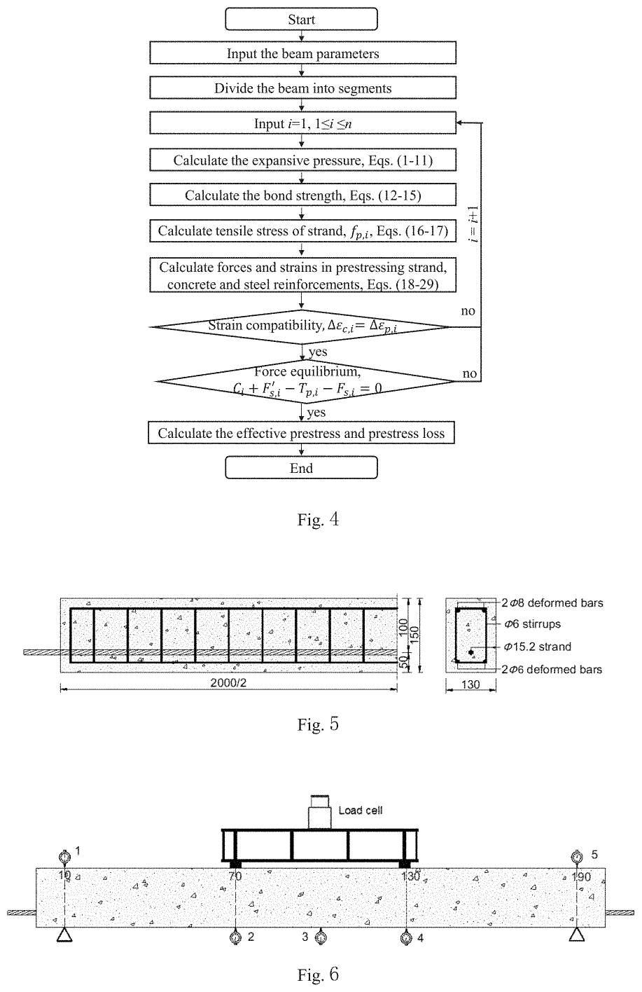

In summary, the present invention provides the method for predicting the prestress loss after concrete cracking along strand, which can comprehensively consider the effects of corrosion-induced concrete cracking and bond degradation. The calculation procedure of corrosion-induced prestress loss is as follows: firstly, corrosion-induced cracking and bond degradation are evaluated considering corrosion effect; secondly, the stress variation in corroded strand at each segment is calculated by equation (17), Based on the step-by-step accumulation, the strand stress increment is obtained by equation (16); thirdly, the accumulation process is terminated when the stresses of prestressing strand, concrete and steel reinforcements in the beam section satisfy the strain compatibility and force equilibrium equations; finally, the effective prestress in the corroded strand can be evaluated by equation (18). The effective prestress in un-corroded strand can be obtained by setting the strand corrosion loss to zero in the above procedure. The prestress loss is equal to the effective prestress in un-corroded strand minus that in corroded strand. FIG. 4 is the calculation flow chart of corrosion-induced prestress loss.

To evaluate the prestress loss in corroded pre-tensioned concrete beams under to various prestress, six concrete beams were designed and fabricated with a rectangular section of 130×150 mm, and a length of 2000 mm. The bottom of the test beam was reinforced with a 15.2 mm diameter 7-wire steel strand and two 6 mm diameter deformed bars of HRB400. The top of the test beam was reinforced with two 8 mm diameter deformed bars of HRB400. The yield strength and ultimate strength of strand were 1830 MPa. and 1910 MPa, respectively. The yield strength and ultimate strength of deformed bars were 400 MPa and 540 MPa, respectively. The thicknesses of concrete cover for ordinary bars and strand were 30 mm and 42.4 mm, respectively. 6 mm diameter stirrups of HPB235 with 100 mm spacing were used in the beams. The 28-day uniaxial compressive strength of the concrete was 44.1 MPa. FIG. 5 illustrates detailed dimension of test beams.

To investigate the variation of prestress loss under various prestress and corrosion degrees, the test beams were divided into groups A and B according to different corrosion times. The corrosion times of groups A and B were 15 and 20 days, respectively. The test beams in each group had different stress states. Taking group A as an example, the prestress in PA1, PA2 and PA3 were 0.25fₚ, 0.5fₚ, and 0.75fₚ, respectively, where fₚ was 1860 Pa. The electrochemical method was employed to accelerate strand corrosion. To clarify the effect of strand corrosion on prestress loss alone, the steel reinforcements were protected with the epoxy resin to prevent corrosion. Direct current was impressed on the strand using a potentiostat, and the corrosion current in the entire process was 0.1 A. A four-point flexural test was performed to obtain the load-deflection curves of corroded specimens, as shown in FIG. 6. The specimen had a clear span of 1800 mm and a bending span of 600 mm. After the loading test, the corrosion degree of strand was evaluated by the average mass loss. Table 1 shows the average mass loss of each test beam.

TABLE 1
Summary of test and theoretical results

| Beam number | PA1 | PA2 | PA3 | PB1 | PB2 | PB3 |
|---|---|---|---|---|---|---|
| Initial prestress | 0.25fₚ | 0.5fₚ | 0.75fₚ | 0.25fₚ | 0.5fₚ | 0.75fₚ |
| ρ (%) | 7.47 | 8.35 | 9.41 | 11.52 | 12.48 | 14.69 |
| F_{c} (kN) | 8.5 | 11.5 | 14.0 | 7.0 | 9.5 | 11.5 |
| F_{e, t} (kN) | 52.6 | 89.7 | 120.7 | 37.5 | 56.4 | 74.0 |
| f_{e, t} (MPa) | 409.0 | 704.1 | 958.5 | 304.9 | 463.6 | 624.0 |
| Test prestress loss | 12.0 | 24.3 | 31.3 | 34.4 | 50.2 | 55.3 |
| ratio (%) | | | | | | |
| Theoretical prestress | 17.7 | 27.3 | 33.7 | 35.6 | 52.4 | 59.3 |
| loss ratio (%) | | | | | | |

Note:
ρ is the average mass loss of strand, F_{c} is the cracking load, F_{e, t} is the test effective prestressing force, and f_{e, t} is the test effective prestress.

During the load testing, the tensile stress at the bottom of beam is affected by self-weight, effective prestress and applied load. Cracks may appear at the bottom of the beam once the tensile stress exceeds the concrete tensile strength. The critical condition of concrete cracking can be expressed as an equation (31)

$${f}_{t} = {f}_{p , η} {A}_{p} ( η ) ( \frac{1}{{A}_{c}} + \frac{{e}_{p}}{{I}_{c}} {y}_{b} ) - \frac{{M}_{s}}{{I}_{c}} {y}_{b} - \frac{{M}_{c}}{{I}_{c}} {y}_{b}$$

where f_{p,η} is the effective prestress in the corroded strand; Aₚ(η) is the residual cross-sectional area of the corroded strand; y_{b} is the distance from the neutral axis to the bottom of the beam; Mₛ is the moment due to beam's weight; M_{c} is the cracking moment; I_{c} is the inertia moment of the gross section of damaged concrete.

Based on the cracking load and corrosion loss, the effective prestress and prestress loss in corroded pre-tensioned concrete beams can be evaluated by equation (31). The relevant results are given in Table 1. The prestress loss ratio in Table 1 is defined as the ratio of the prestress loss in the corroded strand to the effective prestress in the un-corroded strand.

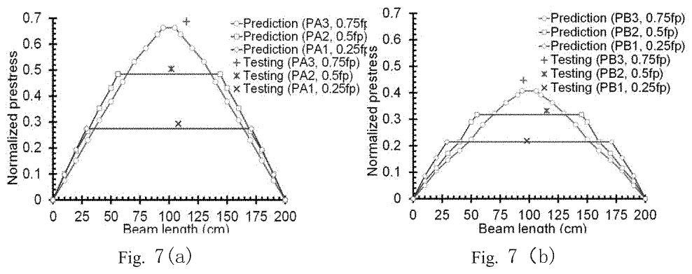

The experimental results are predicted by the method proposed by the present invention. FIGS. 7(a) and 7(b) illustrate predicted and tested values of effective prestress. The standard prestress in the figures is defined as the ratio of the effective prestress of corroded strand to 0.75fₚ. From FIGS. 7(a) and 7(b), it can be found that the average prediction error between the predicted and tested values is 4.8%. Additionally, Table 1 gives the theoretical and experimental prestress loss ratio, which are relatively close, proving the validity of the calculation method. The above analysis shows that the prestress loss prediction method proposed by the present invention can reasonably predict the prestress loss in corroded pre-tensioned concrete beams.

The technical principles of the present invention have been described above in connection with specific embodiments. The descriptions are merely illustrative of the principles of the present invention and are not to be construed as limiting the scope thereof. Based on the explanation herein, those skilled in the art can devise various other embodiments of the present invention without departing from the scope thereof.

What is claimed is:

1. A method for predicting prestress loss after concrete cracking along a strand, comprising steps of:
(1) predicting corrosion-induced concrete cracking, specifically comprising: determining geometric parameters according to specimens details; simulating corrosion-induced cracking by a thick-wall cylinder theory; predicting an expansive pressure with a residual tensile stress by cracked concrete and a confining stress by un-cracked concrete during a concrete cracking process;
(2) analyzing bond strength degradation of a corroded strand, specifically comprising: establishing expressions of an adhesion stress, a confinement stress and a expansive pressure at a strand-concrete interface; considering influence of strand corrosion on the above factors, and then calculate a bond strength of the corroded strand;
(3) evaluating corrosion-induced prestress loss, specifically comprising: discretizing a pre-tensioned concrete member into several segments to analyze stress variation in the corroded strand; considering effects of concrete cracking and bond degradation, an effective prestress in the corroded pre-tensioned concrete member is evaluated with strain compatibility and force equilibrium equations, and then corrosion-induced prestress loss is obtained.

2. The method, as recited in claim 1, wherein in the step (1), during a corrosion-induced concrete cracking process, the expansive pressure is calculated as:
before cover cracking, the expansive pressure is balanced by the residual tensile stress by the cracked concrete and the confining stress by the un-cracked concrete; the expansive pressure P_{c} at the strand-concrete interface is expressed as an equation (1):
P_{c}R₀=PᵤRᵤ+∫_{R}_{₀}^{R}^{ᵤ}σ_{θ}(r)dr
wherein R₀ is a radius of an un-corroded wire, Pᵤ is the expansive pressure at an interface between cracked and un-cracked regions, Rᵤ is a radius of the cracked region, r is a position of the cracked region, and σ_{θ}(r) is a hoop stress of the cracked concrete;
after cover cracking, the expansive pressure is resisted by a residual tensile strength by the cracked concrete; the expansive pressure P_{c} at the strand-concrete interface is expressed as an equation (2):
P_{c}R₀=∫_{R}_{₀}^{R}^{_{c}}σ_{θ}(r)dr.

3. The method, as recited in claim 1, wherein in the step (2), the bond strength of the corroded strand is calculated as:
the bond strength of the corroded strand is attributed to an adhesion stress, a friction stress, and the expansive pressure at the strand-concrete interface, which is expressed as an equation (3):
τ_{η}=τₐ+τ_{b}+τ_{c}
wherein τ_{η} is a bond stress of the corroded strand, τₐ is a bond stress induced by the expansive pressure, τ_{b} is the adhesion stress of the corroded strand, and τ_{c} is the confinement stress from surrounding concrete;
the bond stress induced by the expansive pressure is expressed as an equation (4):
τₐ=k_{c}p_{c}
wherein k_{c} is a friction coefficient between the corroded strand and the cracked concrete;
the adhesion stress of the corroded strand is expressed as an equation (5):
$${τ}_{b} = \frac{k {A}_{r} [ cot δ + tan ( δ + θ ) ]}{π D {s}_{r}} {f}_{c o h}$$
wherein k is a number of transverse ribs, Aᵣ is a rib area in a plane at right angles to a strand axis, D is a strand diameter, δ is a rib orientation, θ is a friction angle between the strand and concrete, sᵣ is a rib spacing, and f_{coh} is a coefficient of the adhesion stress;
the confinement stress from the surrounding concrete is given as an equation (6):
$${τ}_{c} = \frac{k {C}_{r} tan ( δ + θ )}{π} {p}_{x}$$
wherein Cᵣ is a shape factor constant of the transverse ribs, and pₓ is a maximum pressure at bond failure.

4. The method, as recited in claim 1, wherein in the step (3), the effective prestress in the corroded pre-tensioned concrete members is calculated as:
one half of a beam is discretized into several segments from 1 to n to analyze the stress variation in the corroded strand, and for an arbitrary segment i, the stress of the corroded strand f_{p,i} is written as an equation (7):
f_{p,i}=f_{p,i+1}−Δf_{p,i}
wherein Δf_{p,i} is a local stress variation in the corroded strand at the segment i, 1≤i≤n;
the local stress variation in the corroded strand Δf_{p,i} at the segment i is given as an equation (8):
$$Δ {f}_{p , i} = \frac{8 π {R}_{ρ , i}}{{A}_{p , i} ( η )} {τ}_{η} {l}_{i}$$
wherein lᵢ is a segment length; A_{p,i}(η) is a residual cross-sectional area of the corroded strand at the segment i, and R_{ρ,i} is a residual radius of a corroded wire at the segment i;
for corroded pre-tensioned concrete structures, the strand prestress at a beam end is zero, which is f_{p,1}=0; the tensile stress f_{p,i} of the corroded strand at the segment i is expressed as an equation (9):
$${f}_{p , i} = {\sum }_{1}^{i - 1} Δ {f}_{p , i - 1}$$
a tension force T_{p,i} of the corroded strand at the segment i is expressed as an equation (10):
T_{p,i}=f_{p,i}A_{p,i}(η)
after corrosion, a strand strain change Δε_{p,i} is expressed as an equation (11):
$$Δ {ɛ}_{p , i} = \frac{{T}_{p i}}{{E}_{p} {A}_{p}} - \frac{{T}_{p , i}}{{E}_{p} {A}_{p , i} ( η )}$$
wherein Tₚᵢ is a prestressing force of the un-corroded strand at the segment i, and Eₚ is a elastic modulus of the strand;
an internal stress of the corroded strand gradually increases along a member direction until the effective prestress is reached; when the stress of the corroded strand reaches the effective prestress, a concrete strain change at the strand position equals to the strand strain change Δε_{p,i} so that the strain compatibility is maintained, which is expressed as an equation (12):
Δε_{c,i}=Δε_{p,i}
when the stress of the corroded strand reaches the effective prestress, forces of a prestressing strand, the concrete and steel reinforcements satisfy an equilibrium equation (13):
Cᵢ+F′_{s,i}−T_{p,i}−F_{s,i}=0
wherein Cᵢ is a total force of the concrete at the segment i, and F_{s,i} and F′_{s,i} are forces of the steel reinforcements in tension and compression zones at the segment i, respectively;
considering effects of concrete cracking and bond degradation factors, the effective prestress in the corroded pre-tensioned concrete member is calculated with the strain compatibility and force equilibrium equations, and then the corrosion-induced prestress loss is evaluated.
